(19) United States
(12) Patent Application Publication
(10) Pub. No.: US 2009/0292233 A1
(43) Pub. Date: Nov. 26, 2009

(54) ANTI-HUMAN IMMUNODEFICIENCY VIRUS SURROGATE TARGET AGENT TECHNOLOGY FILTER INTENDED TO TERMINATE T-HELPER CELLS INFECTED WITH THE HUMAN IMMUNODEFICIENCY VIRUS CIRCULATING IN BLOOD

(76) Inventors: Lane Bernard Scheiber, Annandale, VA (US); Lane Bernaro Scheiber, II, Grosse Ile, MI (US)

Correspondence Address: LANE B. SCHEIBER, 7864 OAK RIVER DRIVE, GROSS ILE, MI, 48138, US

(21) Appl. No.: 12/123,494

(22) Filed: May 20, 2008

(51) Int. Cl. A61M 1/34

(52) U.S. Cl. 604/5.02; 604/6.09; 604/57; 210/321.6

(57) ABSTRACT

The Human Immunodeficiency Virus posses a significant threat to the world's population. Current strategies have not been adequate to contain and eradicate this deadly viral infection. HIV utilizes a T-Helper cell as a host to generate replicas of itself. Reversing HIV's own biologically deadly tactics and developing blood filtering techniques that incorporate filter mediums that engage the cell-surface receptors uniquely located on the surface of a T-Helper cell infected with the HIV genome can lead to terminating the infected T-Helper cells. Filter mediums possessing cell-surface receptors intended to terminate infected T-Helper cells by triggering apoptosis, is an effective means to eliminate HIV's host cells and thus provides a valuable strategy to prevent and treat AIDS. Similar techniques can be utilized to terminate other types of cells that act as hosts for pathogens as well as terminating cancer cells.

### CROSS-REFERENCE TO RELATED APPLICATIONS

None.

### STATEMENT REGARDING SPONSORED RESEARCH OR DEVELOPMENT

Not applicable.

### REFERENCE TO SEQUENCE LISTING, A TABLE, OR COMPUTER LISTING COMPACT DISC APPENDIX

Not applicable.

©2008 Lane B. Scheiber and Lane B. Scheiber II. A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical device that is utilized to filter the blood of a patient infected with the Human Immunodeficiency Virus with the intention of terminating T-Helper cells infected with the Human Immunodeficiency Virus genome.

2. Description of Background Art

It is estimated by the Center for Disease Control that in the United States 55,000 to 60,000 new cases of Human Immunodeficiency Virus (HIV) are occurring each year. It is thought that there are 900,000 people currently infected with HIV in the United States, with many victims not aware that they have contracted the virus. Further, it has been estimated that the Human Immunodeficiency Virus (HIV), the pathogen that causes Acquired Immune Deficiency Syndrome (AIDS), has infected as many as 30-60 million people around the globe.

The presence of HIV was first came to the attention of those in the United States in 1981, when there appeared an outbreak of Kaposi's Sarcoma and Pneumocystis carinii pneumonia in gay men in New York and California. After over twenty-five years of research and investigation, eradicating the ever growing global humanitarian crisis posed by the HIV remains an elusive goal for the medical community. It is estimated the virus has already killed 25 million citizens of this planet.

The Human Immunodeficiency Virus has been previously referred to as human T-Lymphotrophic virus III (HTLV-III), lymphadenopathy-associated virus (LAV), and AIDS-associated retrovirus (ARV). Infection with HIV may occur by the virus being transferred by blood, semen, vaginal fluid, or breast milk. Four major means of transmission of HIV include unprotected sexual intercourse, contaminated needles, breast milk, and transmission from an infected mother to her baby at birth.

HIV is an ingeniously constructed very deadly virus, which represents the most challenging pathogen the worldwide medical community faces to date. Viruses in general, have been difficult to contain and eradicate due to the fact they are obligate parasites and tend not to carry out any biologic functions outside the cell the virus has targeted as its host. A virus when it exists outside the boundaries of a cell is generally referred to as a virion. HIV virions posses several attributes that make them very elusive and difficult to destroy.

Bacterial infections have posed an easier target for the medical community to eradicate from the body. Bacteria generally live and reproduce outside animal cells. Bacteria, like animal cells, carryout biologic functions. A large multi-celled organism such as the human body combats bacterial infections with a combined force of white cells, antibodies, complements and its lymphatic system. White cells circulate the body in search of bacteria. When a white cell encounters a bacterium, the white cell engulfs the bacterium, encapsulates the pathogen, processes the identification of the pathogen and kills the pathogen utilizing acids and destructive enzymes. The white cell then alerts the B-cells of the immune system as to the identity of the intruding bacterium. A subpopulation of B-cells is generated, dedicated to producing antibodies directed against the particular pathogen the circulating white cell encountered and identified. Antibodies, generated by B-cells, traverse the blood and body tissues in search of the bacteria they were designed to repel. Once an antibody encounters a bacterium it is targeted to attack, the antibody attaches to the bacterium's outer wall. The effect antibodies have in coating the outside of a bacterium is to assist the white cells and the other components of the immune system in recognizing the bacterium, so that appropriate defensive action can be taken against the pathogen. Some antibodies, in addition to coating the bacterium, will act to punch holes through the bacterium's outer wall. If the integrity of the bacterium's cell wall is breached, this action generally leads to the death of the bacterium. Complements are primitive protein structures that circulate the blood stream in search of anything that appears consistent with a bacteria cell wall. Complements are indiscriminant. Once the complement proteins locate any form of bacterial cell wall, the complement proteins organize, and much like antibodies, act in concert to punch one or more holes though a bacterium's cell wall to compromise the viability of the bacterium. The lymphatic system is a diffuse network of thin walled vessels that drain excess water from extracellular fluids and join to form the thoracic duct and right lymph duct, which empty into the venous system near the heart. Lymph nodes are present at different locations in the body and screen the fluid transiting the lymphatic system, called lymph, to remove pathogens. Cells in the spleen screen the blood in search of bacteria. When a bacterial pathogen is identified, such as by antibodies coating the surface, the bacterium is taken out of circulation and terminated.

Viruses pose a much different infectious vector to the body's defense system than either bacteria or cellular parasites. Since viruses do not carry out biologic processes outside their host cell, a virus can be destroyed, but they cannot be killed. A virus is simply comprised of one or more external shells and a portion of genetic material. The virus's genetic information is carried in the core of the virus. Antibodies can coat the exterior of a virus to make it easier for the white cells in the body to identify the viral pathogen, but the action of punching holes in the virus's external shell by antibodies or complement proteins does not necessarily kill the virus. Viruses also only briefly circulate in the blood and tissues of the body as an exposed entity. Using exterior probes, a virus hunts down a cell in the body that will act as an appropriate host so that the virus can replicate. Once the virus has found a proper host cell, the virus inserts its genome into the host cell. To complete its life-cycle, the virus's genetic material takes command of cellular functions and directs the host cell to make replicas of the virus.

Once the virus's genome has entered a host cell, the virus is in effect shielded from the body's immune system defense mechanisms. Inside a host cell, the presence of the virus is generally only represented as genetic information incorporated into the host cell's DNA. Once a virus has infected a cell in the body, the presence of the virus can only be eradicated if the host cell is destroyed. Antibodies and complements are generally designed not to attack the autologous tissues of the body. Circulating white cells and the immune cells which comprise lymph nodes and the spleen may or may not recognize that a cell, which has become a host for a virus, is infected with a virus's genome. If the immune system fails to identify a cell that has become infected with a virus, the virus's genetic material can proceed to force the infected cell to make copies of the virus. Since a virus is in essence simply a segment of genetic material, time is of no consequence to the life-cycle of the virus and a virus's genome may be carried for years by the host without a need to activate; such viruses are often termed latent viruses. A virus's genetic material may sit idle in a host cell for an extended period of time until the pathogen's programming senses the time is right to initiate the virus's replication process or an action of the host cell triggers the virus to replicate. The only opportunity for the immune system to destroy a latent virus is when copies of the virus leave the host cell and circulate in the blood or tissues in search of another perspective host cell.

The traditional medical approach to combating infectious agents such as bacteria and cellular parasites, therefore has limited value in managing or eradicating elusive or latent viral infections. Synthetic antibiotics, generally used to augment the body's capacity to produce naturally occurring antibodies against bacterial infections, have little success in combating latent viral infections. Stimulating the body's immune system's recognition of a virus by administering a vaccine also has had limited success in combating elusive viral infections. Vaccines generally are intended to introduce to the body pieces of a bacteria or virus, or an attenuated, noninfectious intact bacteria or virus so that the immune system is able to recognize and process the infectious agent and generate antibodies directed to assist in killing the pathogen. Once the immune system has been primed to recognize an intruder, antibodies will be produced by the immune system in great quantities in an effort to repel an invader. Over time, as the immune system down-regulates its antibody production in response to a lack of detecting the presence of the intruding pathogen, the quantity of antibodies circulating in the blood stream may decrease in number to a quantity that is insufficient to combat a pathogen. Since antibodies have limited value in combating some of the more elusive viruses that hibernate in host cells, vaccines have limited value in destroying latent viruses.

The Human Immunodeficiency Virus demonstrates four factors which make this pathogen particularly elusive and a difficult infectious agent to eradicate from the body. First: the host for HIV is the T-Helper cell. The T-Helper cell is a key element in the immune system's response since it helps coordinate the body's defensive actions against pathogens seeking to invade the body's tissues. In cases of a bacterial infection versus a viral infection, T-Helper cells actively direct which immune cells will rev-up in response to the infectious agent and engage the particular pathogen. Since HIV infects and disrupts T-Helper cells, coordination of the immune response against the virus is disrupted, thus limiting the body's capacity to mount a proper response against the presence of the virus and produce a sufficient action to successfully eradicate the virus.

Second: again, latent viruses such as HIV, have a strategic advantage. When the immune system first recognizes a pathogen and begins to generate antibodies against a particular pathogen, the response is generally robust. Once time has passed and the immune system fails to detect an active threat, the production of antibodies against the particular pathogen diminishes. When HIV infects a T-Helper cell, the viral genome may lay dormant, sometimes for years before taking command of the T-Helper cell's biologic functions. HIV may, therefore, generate a very active initial immune response to its presence, but if the virus sits dormant inside T-Helper cells for months or years, the antibody response to the virus will diminish over time. There may not be an adequate quantity of circulating antibodies to actively engage the HIV virions as they migrate from the T-Helper cell that generated the copies to uninfected T-Helper cells that will serve as a new host to support further replication. If the immune system's response is insufficient during the period while the virus is exposed and vulnerable, it becomes extremely difficult for the body to eradicate the virus.

Third, when replicas of the Human Immunodeficiency Virus are released from their host cell, during the budding process the HIV virion coats itself with an exterior envelope comprised of a portion of the plasma membrane from the T-Helper cell that acted as the host for the virus. A T-Helper cell's plasma membrane is comprised of a lipid bilayer, a double layer of lipid molecules oriented with their polar ends at the outside of the membrane and the nonpolar ends in the membrane interior. The virus thus, in part, takes on an external appearance of a naturally occurring cell in the body. Since the exterior envelope of a HIV virion has the characteristics of a T-Helper cell it is more difficult for the immune system to recognize that it is a pathogen as it migrates through the body in search of another T-Helper cell to infect.

Fourth, the Human Immunodeficiency Virus exhibits a very elusive mode of action which the virus readily utilizes to actively defeat the body's immune system. HIV carries in its genome a segment of genetic material that directs an infected T-Helper cell to create and mount on the surface the plasma membrane a FasL cell-surface receptor. Healthy T-Helper cells carry on the surface of their plasma membrane Fas cell-surface receptors. The Fas cell-surface receptor when engaged by a FasL cell-surface receptor on another cell, initiates apoptosis in the cell carrying the Fas cell-surface receptor. Apoptosis is a biologic process that causes a cell to terminate itself. A T-Helper cell infected with the HIV virus carrying a FasL cell-surface receptor is therefore capable of killing noninfected T-Helper cells that the infected T-Helper cell encounters as it circulates the body. The occurrence of AIDS is therefore propagated not only by the number of T-Helper cells that become incapacitated due to direct infection by HIV, but also by the number of noninfected T-Helper cells that are eliminated by coming in direct contact with infected T-Helper cells.

Acquired Immune Deficiency Syndrome (AIDS) occurs as a result of the number of circulating T-Helper cells declining to a point where the immune system's capacity to mount a successful response against opportunistic infectious agents is significantly compromised. The number of viable T-Helper cells declines either because they become infected with the HIV virus or because they have been killed by encountering a T-Helper cell infected with HIV. When there is an insufficient population of non-HIV infected T-Helper cells to properly combat infectious agents such as Pneumocystis carinii or cytomegalo virus or other pathogens, the body becomes overwhelmed with the opportunistic infection and the patient becomes clinically ill. In cases where the combination of the patient's compromised immune system and medical assistance in terms of synthetic antibiotics intended to combat the opportunistic pathogens, fluids, intravenous nutrition and other treatments are not sufficient to sustain life, the body succumbs to the opportunistic infection and death ensues.

The Human Immunodeficiency Virus locates its host by utilizing probes located on its envelope. The HIV virion has two types of glycoprotein probes attached to the outer surface of its exterior envelope. A glycoprotein is a structure comprised of a protein component and a lipid component. HIV utilizes a glycoprotein 120 (gp 120) probe to locate a CD4 cell-surface receptor on the plasma membrane of a T-Helper cell. The plasma membrane of the T-Helper cell is comprised of a lipid bilayer. Cell-surface receptors are anchored in the lipid bilayer. Once an HIV gp 120 probe has successfully engaged a CD4 cell-surface receptor on a T-Helper cell a conformational change occurs in the gp 120 probe and a glycoprotein 41 (gp 41) probe is exposed. The gp 41 probe's intent is to engage a CXCR4 or CCR5 cell-surface receptor on the plasma membrane of the same T-Helper cell. Once a gp 41 probe on the HIV virion engages a CXCR4 or CCR5 cell-surface receptor, the HIV virion opens an access portal through the T-Helper cell's plasma membrane.

Once the virus has gained access to the T-Helper cell by opening a portal through the cell's outer membrane the virion inserts two positive strand RNA molecules approximately 9500 nucleotides in length. Inserted along with the RNA strands are the enzymes reverse transcriptase, protease and integrase. Once the virus's genome gains access to the interior of the T-Helper cell, in the cytoplasm the pair of RNA molecules are transformed to deoxyribonucleic acid by the reverse transcriptase enzyme. Following modification of the virus's genome to DNA, the virus's genetic information migrates to the host cell's nucleus. In the nucleus, with the assistance of the integrase protein, the virus's DNA becomes inserted into the T-Helper cell's native DNA. When the timing is appropriate, the now integrated viral DNA, becomes read by the host cell's polymerase molecules and the virus's genetic information commands certain cell functions to carry out the replication process to construct copies of the human deficiency virus.

Present anti-viral therapy has been designed to target the enzymes that assist the HIV genome with the replication process. Anti-viral therapy is intended to interfere with the action of these replication enzymes. Part of the challenge of eradicating HIV is that once the virus inserts its genome into a T-Helper cell host, the viral genome may lay dormant until the proper circumstances evolve. The virus's genome may sit idle inside a T-Helper cell for years before becoming activated, causing drugs that interfere with HIV's life cycle to have limited effect on eliminating the virus from the body. Arresting the replication process does not insure that T-Helper cells infected with HIV do not continue to circulate the body killing noninfected T-Helper cells thus causing the patient to progress to a clinically apparent state of Acquired Immune Deficiency Syndrome and eventually succumbing to an opportunistic infection which eventually results in the death of the individual.

The outer layer of the HIV virion is comprised of a portion of the T-Helper cell's outer cell membrane. In the final stage of the replication process, as a copy of the HIV capsid, carrying the HIV genome, buds through the host cell's plasma membrane, the capsid acquires as its outermost shell a wrapping of lipid bilayer from the host cell's plasma membrane. Vaccines are generally comprised of pieces of a virus or bacterium, or copies of the entire virus or bacterium weakened to the point the pathogen is incapable of causing an infection. These pieces of a pathogen or copies of a nonvirulent pathogen prime the immune system such that a vaccine intent is to cause B-cells to produce antibodies that are programmed to seek out the surface characteristics of the pathogen comprising the vaccine. In the case of HIV, since the surface of the pathogen is an envelope comprised of lipid bilayer taken from the host T-Helper cell's plasma membrane, a vaccine comprised of portions of the exterior envelope of the HIV virions might not only target HIV virions, but might also have deleterious effects on the T-Helper cell population. Some antibodies produced to combat HIV infections may not be able to tell the difference between an HIV virion and a T-Helper cell, and such antibodies may act to coat and assist in the elimination of both targets. In such a scenario, since such a vaccine might cause a decline in the number of available T-Helper cells, it is conceivable that a vaccine comprised of portions of the external envelope of HIV virions might paradoxically induce clinically apparent AIDS in a patient that a vaccine has been administered.

It is clear that the traditional approach of utilizing antibiotics or providing vaccines to stimulate the immune system to produce endogenous antibodies, by themselves, is an ineffective strategy to manage a virus as elusive and deadly as HIV. Drugs that interfere with the replication process of HIV generally slow progression of the infection by the virus, but do not necessarily eliminate the virus from the body nor eliminate the threat of the clinical symptoms of AIDS. A new strategy is required in order to successfully combat the threat of HIV.

Dialysis is generally thought of as a means of removing waste products in patients whose kidneys are no longer capable of effectively filtering the blood and eliminating waste from the body. One option immediately available to reduce the load of T-Helper cells infected with the HIV genome circulating in the blood is by engaging them in a filter chamber with a specially constructed filter medium utilizing a reverse logic with regards to how an infected T-Helper cell would engage and kill a non-infected T-Helper cell, and use this action utilized by an infected T-Helper cell to identify T-Helper cells infected by HIV and terminate them. Reducing the number of HIV infected T-Helper cells would forestall, if not prevent, the onset of AIDS by eliminating T-Helper cells acting as hosts for the virus and eliminating the population of T-Helper cells that act to kill healthy T-Helper cells.

As mentioned earlier, HIV carries in its genome a segment of genetic material that directs an infected T-Helper cell to create and mount on its surface a FasL receptor. T-Helper cells carry, on the surface of their cell plasma membrane a Fas receptor. The Fas receptor, when triggered, initiates apoptosis in the cell. Apoptosis is a biologic process that causes a cell to terminate itself. A T-Helper cell infected with the HIV virus is therefore capable of killing noninfected T-Helper cells that the infected T-Helper cell encounters as it circulates the body. The occurrence of AIDS is therefore enhanced not only by the number of T-Helper cells that become incapacitated due to direct infection by the HIV virus, but also by the number of noninfected T-Helper cells that are eliminated by coming in contact with infected T-Helper cells.

Fas receptors and FasL receptors could be affixed to a filter medium composed of lipid bilayer material or any hypoallergenic material that cell-surface receptors could be affixed to the outer surface. The lipid bilayer material or the hypoallergenic surface material could be in the shape of a sheet, a strip or a sphere. A sheet could take the shape of a square, a rectangle, or the ends could be attached and the sheet could take the shape of a cylinder. A strip could take the shape of a long thin strand where the length is much greater in dimension than the width, or the shape of a coil, or if the ends are attached the strip could take the shape of a ring or circle. The sphere could take the shape of a ball or a cylinder or an ellipsoid.

A lipid bilayer filter medium or a hypoallergenic filter medium with Fas and FasL cell-surface receptors could be used as a filter medium in the filter chamber described in this text. Inside a filter chamber, as blood passed through the chamber, a Fas receptor affixed to the surface of the filter medium would engage a FasL receptor located on an infected T-Helper cell. Once the FasL receptor on the infected T-Helper cell has been engaged by a Fas receptor affixed to the filter medium, a FasL receptor affixed to the same filter medium would then engage a Fas cell-surface receptor on the same infected T-Helper cell. When the filter medium's FasL receptor engages a Fas cell-surface receptor on an infected T-Helper cell this action would cause apoptosis to be triggered in the infected T-Helper cell. Triggering apoptosis in an HIV infected T-Helper cell will cause the cell to kill itself. By terminating HIV infected T-Helper cells, the HIV infection could be prevented from proceeding because by terminating the host cells utilized by HIV, HIV would not be able to replicate itself and HIV virions would no longer emerge to infect additional T-Helper cells.

Constructing a virus-like structure, with the surface characteristics of a virus, that has affixed to its exterior cell-surface receptors intended to engage a T-Helper cell, is referred to as a Scientifically Modulated And Reprogrammed Target (SMART) virus. Such a structure could be simply a sphere of lipid bilayer material with cell-surface receptors attached to the outer surface as described previously, or such structures may carry a filler substance in order to maintain the integrity of the shape of the structure. As the size of the spheres comprised of lipid bilayer material is increased, as needed to be utilized as a filter medium, an inert filler substance may be needed to be placed inside the sphere in order for the sphere to retain their spherical shape. Copies of such a SMART virus could be placed in a filter chamber. Such a filler substance inside the virus-like structure may be represented by a protein or a genetic material that would serve no useful purpose other than acting as a filler. The diameter of the SMART virus could be increased to a size larger than the naturally occurring T-Helper cell to facilitate containing the SMART virus inside the filter chamber as the blood passes through the filter chamber. The SMART virus would be available and remain within the walls of the chamber to engage T-Helper cells as the blood transits through the filter chamber.

Bilayer lipid sheets, hypoallergenic surfaces, scientifically modified and reprogrammed treatment (SMART) viruses otherwise known as virus-like structures, can all be created to act as a filter medium and possess on their surface both Fas and FasL cell-surface receptors. These filter mediums can be fashioned such that the Fas receptor could be physically more prominent than the FasL receptor. The Fas receptor on the surface of a filter medium can be physically mounted further out from the FasL receptor, or in a manner likened as to how HIV is constructed with the gp 120 and gp 41 probes, the Fas receptor may hide the FasL receptor. On the surface of the naturally occurring HIV virion the gp 120 probe covers the gp 41 probe. When the gp 120 probe engages a CD4 cell-surface receptor a conformation change occurs in the gp 120 probe such that the gp 41 probe becomes exposed so that the gp 41 probe can engage either a CXCR4 or CCR5 cell-surface receptor located on the surface of a healthy T-Helper cell. On the surface of a filter medium the Fas receptor would be constructed to be engaged first by an infected T-Helper cell, and once engaged, the FasL receptor affixed to the filter medium would become available to engage a Fas receptor located on an infected T-Helper cell.

Cell-surface receptors are comprised of a protein portion and a lipid portion. The protein portion acts as the receptor. The lipid portion acts as the anchor to fix the cell-surface receptor into the lipid bilayer on the surface of a cell or on the surface of a HIV virion. The lipid portion of the cell-surface receptor could be altered to adjust the distance the protein portion of the cell-surface receptor is physically from the surface of the lipid bilayer. By adjusting the construction, thereby increasing the length of the lipid portion of the Fas cell-surface receptor, the Fas cell-surface receptor affixed to the filter medium could be fashioned to be engaged before a FasL cell-surface receptor affixed to the filter medium is engaged. Alternately the protein portion of the cell-surface receptor closest to the lipid portion of the cell-surface receptor could be lengthened such that increasing the length of the protein portion of the Fas cell-surface receptor, the Fas cell-surface receptor affixed to the filter medium could be fashioned to be engaged before a FasL cell-surface receptor affixed to the filter medium is engaged. Alternatively, the lipid or protein portion of the FasL cell-surface receptor affixed to the surface of a filter medium could be shortened such that the Fas cell-surface receptor affixed to the filter medium is engaged before a FasL cell-surface receptor affixed to the filter medium could be engaged. The intention of positioning the Fas and FasL cell-surface receptors as described is to evoke the action of the T-Helper cells infected with Human Immunodeficiency Virus genome engaging said cell-surface receptors found on the surface of said filter medium in the manner described will trigger apoptosis inside the T-Helper cells infected with the Human Immunodeficiency Virus genome for the purpose of terminating T-Helper cells infected with the Human Immunodeficiency Virus genome, but because the Fas cell-surface receptor affixed to the filter medium must be engaged before a FasL cell-surface receptor affixed to the filter medium can be engaged, T-Helper cells not infected with HIV and other cells expressing a Fas cell-surface receptor will not be harmed by transiting through the described medical filter device.

This technology has a much broader range of beneficial medical treatment uses beyond simply eliminating T-Helper cells infected with the HIV genome from the blood. All cells have cell-surface receptors. Many cells in the body have affixed to their surface cell-surface receptors that are unique to the specific type of cell. By utilizing the concept of mounting on a filter medium specialized cell-surface receptors that engage a unique cell-surface receptor located on the surface of a specific target cell circulating in the blood, specific target cells which also express a Fas cell-surface receptor on their surface can be caused to be terminated, with such action resulting a beneficial medical outcome. To accomplish this, on a filter medium would be affixed specialized cell surface receptors and FasL cell-surface receptors. The specialized cell-surface receptors would be constructed to be more prominent than the FasL cell-surface receptors, such that the specialized cell-surface receptors would be engage before a FasL cell-surface receptor could be engaged. As blood transits through a filter chamber with the above-mentioned filter medium contained inside, specific target cells would come in contact with the filter medium. Once the unique cell-surface receptor located on the specific target cell engaged a specialized cell-surface receptor affixed to the filter medium, then the FasL cell-surface receptor affixed to the filter medium would engage a Fas cell-surface receptor on the specific target cell. By the action of the FasL cell-surface receptor affixed to the filter medium engaging the Fas cell-surface receptor located on the specific target cell, the signal of apoptosis would be triggered in the specific target cell and the specific target cell would terminate itself. By physically constructing the cell-surface receptors on the filter medium such that the specialized cell-surface receptor must be engaged before the FasL cell-surface receptors can be engaged, facilitates that cells that do not carry the unique cell-surface receptor such as affixed to the surface of the specific target cell, will not be harmed by transiting through the medical filter device.

Such a filter device could be used to treat patients with cancers such as various forms of leukemia. Forms of leukemia flood the circulating blood with numerous leukemic cells. The presence of this abundance of leukemic cells interferes with the function of normal blood cells and blood plasma. For several forms of leukemia such as chronic lymphocytic Leukemia (CLL), the medical treatment approach has provided very limited benefit. Often for CLL patients, available chemotherapy treatment produces side effects that are worse than the effects of the CLL on the patient. Patients with CLL often suffer for years with the leukemia adversely affecting their bodies. A filter medium designed to utilize specialized cell-surface receptors to engage one or more unique cell-surface receptors on a leukemic cell, which once a leukemic cell would be engaged, then FasL receptors on the filter medium could engage one or more Fas cell-surface receptors on the leukemic cell, the result of which would be the leukemic cell would receive the signal that would tell the leukemic cell to terminate itself. Successfully terminating leukemic cells would reduce the quantity of circulating leukemic cells. A reduction in the quantity of circulating leukemic cells would improve beneficial performance of blood in such patients.

Other medical conditions such as parasitic infections, where a parasite has infected a blood cell and the infected blood cell expresses a unique cell-surface receptor and a Fas cell-surface receptor, such an infected cell could be terminated by a similar strategy as described above.

Any cell that is harmful to the body that circulated in the blood, that carries a unique cell-surface receptor and carries a Fas cell-surface receptor could be terminated by the above-mentioned strategy and thus eliminated from the body, to produce a medically beneficial effect.

A filter device could be fashioned to be placed inside the body to act to continuously filter the blood and terminate cancer cells of a particular type that transit the blood stream. Concerns regarding metastatic cancer could be treated by such a device. Metastatic cancer occurs when cells from a primary cancer site in the body leave the primary site and migrate through the blood and develop one or more secondary or satellite sites of cancer in the body. Once a person is found to have a cancer, or in an individual who is at high risk for developing metastatic cancer, a filter could be placed in a blood vessel, a vein or an artery, and such a filter would continuously filter the blood and engage only the cancer cells that transit through the filter allowing all other blood cells to pass through unharmed. Knowing the type of cancer the filter was created to engage, the filter medium could be created to have fixed to its surface specialized cell-surface receptors that engage unique cell-surface receptors on the surface of the specific type of targeted cancer cell. A cancer cell of a specific type that transits through the filter would be engaged by the specialized cell-surface receptor affixed to the filter medium. Once the specific type of cancer cell is engaged, a FasL cell-surface receptor located on the filter medium would engage a Fas cell-surface receptor located on the cancer cell and such an action would trigger apoptosis in the cancer cell and the cancer cell would terminate itself. The filter device would not need to be renewed and the filter device would not clog blood flow because the cancer cells would exit the filter chamber, cell death would ensue at a later time and such cells would then eventually be removed from circulation and re-absorb by the body. Such a device could be fashioned to be inserted in a vessel of the lymphatic system to constantly filter lymph and engage and terminate infected T-Helper cells or other specific target cells as they transit through the filter device. Cancers often spread locally from a primary site through the lymphatic system and such a filter device could be inserted into a vessel of the lymphatic system downstream to a primary site of cancer to screen and terminate any cancer cells that had migrated from the primary site of the cancer.

Similarly, a patient infected with HIV or patient at high risk for infection with HIV, could have a small filter placed inside their body. Such a filter could continuously screen the blood with the intention that any T-Helper cell infected with the HIV genome that is expressing both FasL and Fas cell-surface receptors that transit though the filter device will be engaged. Once a Fas cell-surface receptor affixed to the filter medium inside the filter device has been engaged by a FasL cell-surface receptor located on an infected T-Helper cell, a FasL cell-surface receptor affixed to the filter medium would then engage a Fas cell-surface receptor on the infected T-Helper cell. When the filter medium's FasL cell-surface receptor engages a Fas cell-surface receptor located on an infected T-Helper cell this action will cause apoptosis to be triggered in the infected T-Helper cell. Triggering apoptosis in a cell will cause the cell to kill itself. By this means infected T-Helpers, the host cell of HIV, can be continuously eliminated from the body. Such a filter would not need to be renewed and the filter would not clog blood flow because the infected T-Helper cells would exit the filter chamber, cell death would ensue at a later time and such cells would be removed from circulation and be re-absorb by the body.

### BRIEF SUMMARY OF THE INVENTION

A T-Helper cell infected with the Human Immunodeficiency Virus genome express FasL cell-surface receptors and Fas cell-surface receptors on its exterior surface. This medical filter device is intended to engage T-Helper cells circulating in the blood. The filter medium contained inside the filter device has affixed to its surface Fas and FasL cell-surface receptors. The Fas cell-surface receptors affixed to the surface of the filter medium located inside the device are more prominent than the FasL cell-surface receptors located on the surface of the filter medium. As infected T-Helper cells transit the filter device, the FasL cell-surface receptors they carry will engage Fas cell-surface receptors affixed to the filter medium. Once a FasL cell-surface receptor on an infected T-Helper cell has engaged a Fas cell-surface receptor affixed to the filter medium, a FasL cell-surface receptor affixed to the filter medium will engage a Fas cell-surface receptor located on the surface of the infected T-Helper cell. By having a FasL cell-surface receptor affixed to the filter medium engage a Fas cell-surface receptor located on an infected T-Helper cell, the process of apoptosis is triggered in the T-Helper cell carrying the Fas cell-surface receptor. Activating the process of apoptosis in a cell leads to cell death. By terminating T-Helper cells that are infected with the Human Immunodeficiency Virus genome leads to an effective means for averting AIDS. A similar strategy utilizing specialized cell-surface receptors and FasL cell-surface receptors can be employed to terminate specific target cells such as cancer cells and cells harboring parasites for the purpose of achieving a beneficial medically therapeutic outcome. A medical device constructed with a filter medium can be fashioned to be placed in a vessel of the lymphatic system which would continuously screen lymph and terminate cancer cells or other target cells to result in a medically beneficial outcome.

### DETAILED DESCRIPTION OF THE INVENTION

The invention described herein is intended to terminate T-Helper cells infected with Human Immunodeficiency Virus and other specific target cells such as cancer cells and host cells harboring parasites, as they circulate in fluid such as blood or lymph. The medical device may be used in an intermittent dynamic process such as where blood is actively removed from an individual, the blood transits through one or more filtering devices and the cleansed blood is then returned to the same individual. The medical device may be used in a process which is more static, where a specific quantity of blood is removed from one individual, the blood products transit through one or more filtering devices and this now cleansed blood or separate blood products are, at a later time, infused into one or more individuals in need of such blood products. The medical device may be used in a continuous dynamic process, where a filter device is inserted in a blood vessel or a lymphatic vessel inside the body, which such a medical device constantly acts to filter the blood or lymph and engage and terminate infected T-Helper cells or other specific target cells as they transit through the filter device.

The medical device described herein, intended to terminate T-Helper cells infected with HIV genome as they exist in blood, is comprised of a chamber, where blood is introduced into the chamber at one location, the blood comes into contact with a filter medium, the blood exits the chamber at a different location than where the blood plasma entered the chamber. The filter medium inside the filter chamber may be comprised of several different materials and designs. The filter medium is intended to make available cell-surface receptors including Fas and FasL for T-Helper cells infected with HIV genome to engage. The filter medium may be comprised of a quantity of lipid bilayer sheets which are comprised of similar materials as found existing as the outer membrane of a T-Helper cell, and affixed to the said lipid bilayer sheets are glycoprotein cell-surface receptors including a quantity of Fas cell-surface receptors and FasL cell-surface receptors. Such bilayer sheets may be of any suitable shape which might include such shapes as the shape of a square, the shape of a rectangle, the sheet may be attached to itself to be the shape of a cylinder. The filter medium may be comprised of a quantity of lipid bilayer strips which are comprised of similar materials as found existing as the outer membrane of a T-Helper cell, and affixed to the said lipid bilayer strips are glycoprotein cell-surface receptors including a quantity of Fas cell-surface receptors and FasL cell-surface receptors. Such strips may be long and thin and may include any suitable shape such as a long thin strand, or the shape of a coil or one end may be attached to another end to form the shape of a ring or circle. The filter medium may be comprised of a quantity of lipid bilayer spheres which are comprised of similar materials as found existing as the outer membrane of a T-Helper cell, and affixed to the said lipid bilayer spheres are glycoprotein cell-surface receptors including a quantity of Fas cell-surface receptors and FasL cell-surface receptors. The shapes of the spheres may include any suitable shape such as the shape of a ball, the shape of cylinder, the shape of an ellipsoid. The filter medium may be comprised of a quantity of virus-like structures with cell-surface receptors to include a quantity of Fas cell-surface receptors and FasL cell-surface receptors. The filter medium may be comprised of any suitable hypoallergenic material, which can be affixed to the surface a quantity of Fas cell-surface receptors and FasL cell-surface receptors or simply the protein portion of the Fas cell-surface receptors and FasL cell-surface receptors. The shape of the hypoallergenic material may include a variety of suitable shapes including the shape of a sheet, shape of a strip or shape of a sphere.

The material to be used to create the walls of such a filter chamber may include any suitable hypoallergenic material such as glass, rigid plastic, a flexible plastic, latex, steel, aluminum or other metal or metal alloy. A tube to carry blood or blood plasma to the filter chamber would be attached to the portal where the blood or blood plasma would enter the filter chamber. A tube would be attached to the portal of the filter chamber where the blood or blood plasma would exit the chamber to carry the filtered blood or blood plasma away from the chamber. The tubing carrying blood or blood plasma to the filter chamber and the tubing carrying blood or blood plasma away from the filter chamber would be comprised of any hypoallergenic material such as a flexible plastic, rigid plastic, a flexible metal or a rigid metal or latex. A porous barrier located at the portal where the blood or blood plasma enters the filter chamber and a porous barrier located at the portal where the blood or blood plasma exits the filter chamber would be comprised of materials such as a flexible plastic, a rigid plastic, a flexible metal or a rigid metal or latex. The said porous barriers are comprised of a quantity of holes, said holes large enough to allow said blood or blood plasma to freely exit said chamber, but said holes are restrictive enough so as to retain said filter medium inside the inner boundaries of said chamber as said blood or blood plasma transits through said chamber. The filter medium contained inside the filter chamber may be free-floating within the inner boundaries of the filter chamber or may be physically fixed to the chamber such that the filter medium does not move freely inside the filter chamber and cannot exit the filter chamber.

Lipid bilayer sheets, strips, spheres can be manufactured and combinations of Fas cell-surface receptors and FasL cell-surface receptors can be affixed to the surface with the entire structure acting as a filter medium. Sheets of any suitable hypoallergenic material can be manufactured and combinations of Fas cell-surface receptors and FasL cell-surface receptors can be affixed to the surface with the structure acting as a filter medium. Sheets of any suitable hypoallergenic material can be manufactured and combinations of the protein portion of the Fas cell-surface receptors and FasL cell-surface receptors attached to the surface of the hypoallergenic surface and made available to engage either glycoprotein probes on HIV or cell-surface receptors on a T-Helper cell, with the structure acting as a filter medium.

To carry out the process to manufacture a virus-like structure, DNA or RNA code that would provide the necessary biologic instructions to generate the general physical outer structures of the virus-like structure, would be inserted into a host. The host may include devices such as a host cell or a hybrid host cell. The host may utilize DNA or RNA or a combination of genetic instructions in order to accomplish the construction of medically therapeutic virus-like structures. In some cases DNA or messenger RNA would be inserted into the host that would be coded to cause the production of cell-surface receptors that would be affixed to the surface of the virus-like structure that would target the glycoprotein probes affixed to the surface of an HIV virion or the FasL and Fas cell-surface receptors on infected T-Helper cells. The copies of the medically therapeutic virus-like structures, upon exiting the host, would be collected, stored and utilized as a filter medium in the described filter chamber as necessary.

The medically therapeutic version of the virus-like structures would be incapable of replication on its own due to the fact that the messenger RNA or DNA that would code for the replication process to produce copies of the virus-like structure would not be present in the virus-like structures.

The medical device intended to terminate T-Helper cells infected with the HIV genome is comprised of a chamber, where blood is introduced into the chamber at one location, the blood comes into contact with a filter medium, the blood exits the chamber at a different location than where the blood entered the chamber. The filter medium inside the filter chamber is fashioned to express on its surface a quantity of Fas cell-surface receptors and FasL cell-surface receptors. The Fas cell-surface receptors are mounted on the surface of the filter medium in a manner that they are to be engaged before the FasL cell-surface receptors can be engaged.

The invention described herein is intended to terminate T-Helper cells infected with the HIV genome from a fluid such as blood. The filtering process may be intermittently dynamic such as blood that is actively removed from an individual, the blood transits through one or more filtering devices and the cleansed blood is then returned to the same individual. In the filtering process as the blood from the individual makes contact with the filter medium inside the filter device terminates T-Helper cells infected with the HIV virus. Blood cleansed of HIV is returned to the same individual.

The filter device may be used in a more static process, where a specific quantity of blood is removed from one individual, the blood products transit through one or more filtering devices and this now cleansed blood or separate blood products are, at a later time, infused into one or more other individuals in need of such cleansed blood products. The blood permanently removed from the first individual makes contact with the filter medium inside the filter device terminates T-Helper cells infected with the HIV virus. Blood removed from the first individual, now cleansed of HIV infected T-Helper cells, is then provided to one or more other individuals requiring such blood.

The medical device may be used in a continuous dynamic process, where a filter device is inserted in a blood vessel inside the body, which constantly acts to constantly filter the blood and engage and terminate infected T-Helper cells as they transit through the filter device.

This technology has a much broader range of beneficial uses beyond just eliminating T-Helper cells infected with the HIV genome from the blood. All cells have surface cell receptors. Many cells in the body have affixed to their surface cell-surface receptors that are unique to the specific type of cell. By utilizing the concept of mounting on a filter medium specialized cell-surface receptors that engage a unique cell-surface receptor located on the surface of a specific target cell circulating in the blood, specific target cells can be caused to be terminated, with such action resulting a beneficial medical outcome. To accomplish this, on a filter medium would be affixed specialized cell surface receptors and FasL cell-surface receptors. The specialized cell-surface receptors would be constructed to be more prominent than the FasL cell-surface receptors, such that the specialized cell-surface receptors would be engaged before a FasL cell-surface receptor could be engaged. As blood transited through a filter chamber with the above-mentioned filter medium contained inside, specific target cells would come in contact with the filter medium. Once the unique cell-surface receptor on the specific target cell engaged a specialized cell-surface receptor on the filter medium, then the FasL cell-surface receptor on the filter medium would engage a Fas cell-surface receptor on the specific target cell. By the action of the FasL cell-surface receptor affixed to the filter medium engaging the Fas cell-surface receptor located on the specific target cell, the signal of apoptosis would be triggered in the specific target cell and the specific target cell would terminate itself. By constructing the cell-surface receptors on the filter medium such that the specialized cell-surface receptor must be engaged before the FasL cell-surface receptor can be engaged, facilitates that cells that do not carry the unique cell-surface receptor such as affixed to the surface of the specific target cell will not be harmed by transiting through the filter device.

Such a filter device could be used to treat patients with cancers such as various forms of leukemia. Forms of leukemia flood the circulating blood with numerous leukemic cells. The presence of this abundance of leukemic cells interferes with the function of normal blood cells and blood plasma. For several forms of leukemia such as chronic lymphocytic Leukemia (CLL), the medical treatment approach has provided very limited benefit. Often for CLL patients, available chemotherapy treatment produces side effects that are worse than the effects of the CLL on the patient. Patients with CLL often suffer for years with the leukemia adversely affecting their bodies. A filter medium designed to utilize specialized cell-surface receptors to engage one or more unique cell-surface receptors on a leukemic cell, which once a leukemic cell would be engaged, then FasL receptors on the filter medium could engage one or more Fas cell-surface receptors on the leukemic cell, the result of which would be the leukemic cell would receive the signal that would tell it to terminate itself. Successfully terminating leukemic cells would reduce the quantity of circulating leukemic cells. A reduction in the quantity of circulating leukemic cells would improve beneficial performance of blood.

Other medical conditions such as parasitic infections, where a parasite has infected a blood cell and the infected blood cell expresses a unique cell-surface receptor and a Fas cell-surface receptor, such an infected cell could be terminated by a similar strategy as described above.

Any cell that is harmful to the body that circulated in the blood, that carries a unique cell-surface receptor and carries a Fas cell-surface receptor could be terminated by the above-mentioned strategy and thus eliminated from the body, to produce a medically beneficial effect.

The medical device may be constructed to exist outside the body and engage in an intermittent dynamic process where blood is actively removed from an individual, the blood transits through one or more filtering devices and the cleansed blood is then returned to the same individual. The medical device may be constructed to exist outside the body and engage in a more static process where a specific quantity of blood is removed from one individual, the blood products transit through one or more filtering chambers and this now cleansed blood or separate blood products are, at a later time, is infused into one or more individuals in need of such blood products. The medical device may be constructed as a device to be inserted in a blood vessel inside the body, which the medical device constantly acts to filter the blood and engage and terminate specific target cells such as infected T-Helper cells, cancer cells, host cells infected by a parasite, as such cells transit through the filter device.

Portions of the lymphatic system can be continuously filtered with such a medical device. Utilizing the concept of mounting on a filter medium specialized cell-surface receptors that engage a unique cell-surface receptor located on the surface of a specific target cell transiting the lymph, specific target cells can be caused to be terminated, with such action resulting a beneficial medical outcome. To accomplish this, on a filter medium would be affixed specialized cell surface receptors and FasL cell-surface receptors. The specialized cell-surface receptors would be constructed to be more prominent than the FasL cell-surface receptors, such that the specialized cell-surface receptors would be engage before a FasL cell-surface receptor could be engaged. As lymph transits through a filter chamber with the above-mentioned filter medium contained inside, specific target cells would come in contact with the filter medium. Once the unique cell-surface receptor on the specific target cell engages a specialized cell-surface receptor on the filter medium, then the FasL cell-surface receptor on the filter medium would engage a Fas cell-surface receptor on the specific target cell. By the action of the FasL cell-surface receptor affixed to the filter medium engaging the Fas cell-surface receptor located on the specific target cell, the signal of apoptosis would be triggered in the specific target cell and the specific target cell would terminate itself. By constructing the cell-surface receptors on the filter medium such that the specialized cell-surface receptor must be engaged before the FasL cell-surface receptor can be engaged, facilitates that cells that do not carry the unique cell-surface receptor such as affixed to the surface of the specific target cell will not be harmed by transiting through the filter device. Such a medical device could be fashioned to be inserted in a vessel of the lymphatic system to constantly filter lymph to engage and terminate infected T-Helper cells or other specific target cells as they transit through the medical device.

### DRAWINGS

None.

What is claimed:

1. A medical device, to terminate T-Helper cells infected with Human Immunodeficiency Virus genome as they circulate through blood, comprised of a chamber:
(a) where blood enters said chamber at one portal,
(b) said blood comes into contact with a filter medium,
(c) said filter medium having cell-surface receptors affixed to its surface,
(d) said blood exits said chamber at a different portal than where said blood entered said chamber,
(e) said filter medium is retained inside said chamber,
whereby said T-Helper cells infected with Human Immunodeficiency Virus genome are intended to come in contact with said cell-surface receptors found on the surface of said filter medium contained inside said chamber,
whereby said T-Helper cells infected with Human Immunodeficiency Virus genome are intended to engage said cell-surface receptors found on the surface of said filter medium contained inside said chamber with the intention of triggering apoptosis inside said T-Helper cells infected with the Human Immunodeficiency Virus genome for the purpose of terminating said T-Helper cells infected with the Human Immunodeficiency Virus genome utilizing the natural process of apoptosis to actuate cell death,
whereby said chamber may be used as a medical device that is inserted into the body or said chamber may be used as a medical device outside the body.

2. The medical device in claim 1 wherein said filter medium selected from the group consisting of a quantity of lipid bilayer sheets, a quantity of lipid bilayer strips, a quantity of lipid bilayer spheres, a quantity of virus-like structures, and a quantity of hypoallergenic surfaces capable of supporting the functional expression of a quantity of said cell-surface receptors.

3. The medical device in claim 1 wherein said filter medium is a quantity of virus-like structures.

4. The medical device in claim 1 wherein said filter medium selected from the group consisting of a quantity of lipid bilayer sheets, a quantity of lipid bilayer strips, and a quantity of lipid bilayer spheres.

5. The medical device in claim 1 wherein said filter medium selected from the group consisting of a quantity of hypoallergenic surfaces fashioned in the shape of a sheet capable of acting as a base on which can be affixed said cell-surface receptors to support the functional expression of a quantity of said cell-surface receptors, a quantity of hypoallergenic surfaces fashioned in the shape of a strip capable of acting as a base on which can be affixed said cell-surface receptors to support the functional expression of a quantity of said cell-surface receptors, and a quantity of hypoallergenic surfaces fashioned in the shape of a sphere capable of acting as a base on which can be affixed said cell-surface receptors to support the functional expression of a quantity of said cell-surface receptors.

6. The medical device in claim 1 wherein said blood is removed from said body, said blood transits said medical device and said blood is returned to said body.

7. The medical device in claim 1 wherein said chamber is fashioned to be inserted into a blood vessel in a body, whereby said medical device continuously filters the blood.

8. The medical device in claim 1 wherein at about the location of said portal where said blood enters said chamber a porous barrier is present comprised of a quantity of holes, said holes are sufficient in the size of their dimensions to allow said blood to freely enter said chamber, but said holes are restrictive enough in the size of their dimensions so as to retain said filter medium inside the inner boundaries of said chamber as said blood transits through said chamber.

9. The medical device in claim 1 wherein at about the location of said portal where said blood exits said chamber a porous barrier is present comprised of a quantity of holes, said holes are sufficient in the size of their dimensions to allow said blood to freely exit said chamber, but said holes are restrictive enough in the size of their dimensions so as to retain said filter medium inside the inner boundaries of said chamber as said blood transits through said chamber.

10. The medical device in claim 1 wherein said chamber is in the shape of a tube where said chamber is fashioned to wrap around onto itself such that one end is affixed to the other end such that said filter medium is present inside said chamber and said filter medium is retained inside said chamber as said blood enters said chamber through said entry portal, transits said chamber while doing so said blood makes contact with the surface of said filter medium, then said blood exits said chamber through said exit portal in said chamber.

11. The medical device in claim 1 wherein an initiative to move said blood through said chamber is executed by roller devices that apply gentle compressive and decompressive forces on the exterior of said chamber to create a flow of said blood through said chamber from said entry portal to said exit portal of said chamber.

12. The medical device in claim 1 wherein said cell-surface receptors are comprised of a quantity of Fas cell-surface receptors and a quantity of FasL cell-surface receptors.

13. The medical device in claim 12 wherein said quantity of Fas cell-surface receptors are positioned on the surface of said filter medium in a manner such that they are to be physically engaged by FasL cell-surface receptors located on said T-Helper cells infected with the Human Immunodeficiency Virus genome before said FasL cell-surface receptors affixed to said filter medium are able to engage Fas cell-surface receptors located on said T-Helper cells infected with the Human Immunodeficiency Virus genome, then once a said Fas cell-surface receptor affixed to said filter medium is engaged by a said FasL cell-surface receptor located on said T-Helper cell infected with the Human Immunodeficiency Virus genome, a quantity of said FasL cell-surface receptors affixed to the surface of the filter medium are allowed the opportunity to engage a quantity of Fas cell-surface receptors located on said T-Helper cell infected with the Human Immunodeficiency Virus genome,
whereby the action of said T-Helper cells infected with Human Immunodeficiency Virus genome engaging a quantity of said Fas cell-surface receptors affixed to the surface of said filter medium followed by engaging a quantity of said FasL cell-surface receptors affixed to the surface of said filter medium in the manner described will trigger apoptosis inside said T-Helper cells infected with the Human Immunodeficiency Virus genome for the purpose of terminating said T-Helper cells infected with the Human Immunodeficiency Virus genome,
whereby the intention that said Fas cell-surface receptor affixed to the filter medium must be engaged before said FasL cell-surface receptor affixed to the filter medium can be engaged, insures that T-Helper cells not infected with the HIV genome as well as other cells expressing a quantity of Fas cell-surface receptors but not expressing a quantity of FasL cell-surface receptors, will not be harmed by transiting through said medical device.

14. The medical device in claim 12 wherein said quantity of Fas cell-surface receptors are physically constructed to facilitate said Fas cell-surface receptors affixed to said filter medium to be physically engaged by FasL cell-surface receptors located on said T-Helper cells infected with the Human Immunodeficiency Virus genome before said FasL cell-surface receptors affixed to said filter medium are able to engage Fas cell-surface receptors located on said T-Helper cells infected with the Human Immunodeficiency Virus genome, then once a said Fas cell-surface receptor affixed to said filter medium is engaged by a said FasL cell-surface receptor located on said T-Helper cell infected with the Human Immunodeficiency Virus genome, a quantity of said FasL cell-surface receptors affixed to said filter medium are allowed the opportunity to engage a quantity of Fas cell-surface receptors located on said T-Helper cell infected with the Human Immunodeficiency Virus genome,
whereby the action of said T-Helper cells infected with Human Immunodeficiency Virus genome engaging a quantity of said Fas cell-surface receptors affixed to the surface of said filter medium followed by engaging a quantity of said FasL cell-surface receptors affixed to the surface of said filter medium in the manner described will trigger apoptosis inside said T-Helper cells infected with the Human Immunodeficiency Virus genome for the purpose of terminating said T-Helper cells infected with the Human Immunodeficiency Virus genome,
whereby the intention that said Fas cell-surface receptor affixed to the filter medium must be engaged before said FasL cell-surface receptor affixed to the filter medium can be engaged, insures that T-Helper cells not infected with the HIV genome as well as other cells expressing a quantity of Fas cell-surface receptors but not expressing a quantity of FasL cell-surface receptors, will not be harmed by transiting through said medical device.

15. The medical device in claim 12 wherein said Fas cell-surface receptors are physically altered such that the lipid portion of said Fas cell-surface receptors is lengthened in a fashion to make the protein portion of said Fas cell-surface receptors affixed to said filter medium more prominent than the protein portion of said FasL cell-surface receptors affixed to said filter medium, to facilitate said Fas cell-surface receptors affixed to said filter medium to be physically engaged by FasL cell-surface receptors located on said T-Helper cells infected with the Human Immunodeficiency Virus genome before said FasL cell-surface receptors affixed to said filter medium are able to engage Fas cell-surface receptors located on said T-Helper cells infected with the Human Immunodeficiency Virus genome, then once a said Fas cell-surface receptor affixed to said filter medium is engaged by a said FasL cell-surface receptor located on said T-Helper cell infected with the Human Immunodeficiency Virus genome, a quantity of said FasL cell-surface receptors affixed to said filter medium are allowed the opportunity to engage a quantity of Fas cell-surface receptors located on said T-Helper cell infected with the Human Immunodeficiency Virus genome,
whereby the action of said T-Helper cells infected with Human Immunodeficiency Virus genome engaging a quantity of said Fas cell-surface receptors affixed to the surface of said filter medium followed by engaging a quantity of said FasL cell-surface receptors affixed to the surface of said filter medium in the manner described will trigger apoptosis inside said T-Helper cells infected with the Human Immunodeficiency Virus genome for the purpose of terminating said T-Helper cells infected with the Human Immunodeficiency Virus genome,
whereby the intention that said Fas cell-surface receptor affixed to the filter medium must be engaged before said FasL cell-surface receptor affixed to the filter medium can be engaged, insures that T-Helper cells not infected with the HIV genome as well as other cells expressing a quantity of Fas cell-surface receptors but not expressing a quantity of FasL cell-surface receptors, will not be harmed by transiting through said medical device.

16. The medical device in claim 12 wherein said quantity of FasL cell-surface receptors are physically constructed to facilitate said Fas cell-surface receptors affixed to said filter medium to be physically engaged by FasL cell-surface receptors located on said T-Helper cells infected with the Human Immunodeficiency Virus genome before said FasL cell-surface receptors affixed to said filter medium are able to engage Fas cell-surface receptors located on said T-Helper cells infected with the Human Immunodeficiency Virus genome, then once a said Fas cell-surface receptor affixed to said filter medium is engaged by a said FasL cell-surface receptor located on said T-Helper cell infected with the Human Immunodeficiency Virus genome, a quantity of said FasL cell-surface receptors affixed to said filter medium are allowed the opportunity to engage a quantity of Fas cell-surface receptors located on said T-Helper cell infected with the Human Immunodeficiency Virus genome,
whereby the action of said T-Helper cells infected with Human Immunodeficiency Virus genome engaging a quantity of said Fas cell-surface receptors affixed to the surface of said filter medium followed by engaging a quantity of said FasL cell-surface receptors affixed to the surface of said filter medium in the manner described will trigger apoptosis inside said T-Helper cells infected with the Human Immunodeficiency Virus genome for the purpose of terminating said T-Helper cells infected with the Human Immunodeficiency Virus genome,
whereby the intention that said Fas cell-surface receptor affixed to the filter medium must be engaged before said FasL cell-surface receptor affixed to the filter medium can be engaged, insures that T-Helper cells not infected with the HIV genome as well as other cells expressing a quantity of Fas cell-surface receptors but not expressing a quantity of FasL cell-surface receptors, will not be harmed by transiting through said medical device.

17. The medical device in claim 12 wherein said FasL cell-surface receptors are physically altered such that the lipid portion of said receptor is shortened in a fashion to make the protein portion of said FasL cell-surface receptor less prominent than the protein portion of said Fas cell-surface receptor to facilitate said Fas cell-surface receptors affixed to said filter medium to be physically engaged by FasL cell-surface receptors located on said T-Helper cells infected with the Human Immunodeficiency Virus genome before said FasL cell-surface receptors affixed to said filter medium are able to engage Fas cell-surface receptors located on said T-Helper cells infected with the Human Immunodeficiency Virus genome, then once a said Fas cell-surface receptor affixed to said filter medium is engaged by a said FasL cell-surface receptor located on said T-Helper cell infected with the Human Immunodeficiency Virus genome, a quantity of said FasL cell-surface receptors affixed to said filter medium are allowed the opportunity to engage a quantity of Fas cell-surface receptors located on said T-Helper cell infected with the Human Immunodeficiency Virus genome,
whereby the action of said T-Helper cells infected with Human Immunodeficiency Virus genome engaging a quantity of said Fas cell-surface receptors affixed to the surface of said filter medium followed by engaging a quantity of said FasL cell-surface receptors affixed to the surface of said filter medium in the manner described will trigger apoptosis inside said T-Helper cells infected with the Human Immunodeficiency Virus genome for the purpose of terminating said T-Helper cells infected with the Human Immunodeficiency Virus genome,
whereby the intention that said Fas cell-surface receptor affixed to the filter medium must be engaged before said FasL cell-surface receptor affixed to the filter medium can be engaged, insures that T-Helper cells not infected with the HIV genome as well as other cells expressing a quantity of Fas cell-surface receptors but not expressing a quantity of FasL cell-surface receptors, will not be harmed by transiting through said medical device.

18. A medical device, to terminate specific target cells as they circulate through blood, comprised of a chamber:
(a) where blood enters said chamber at one portal,
(b) said blood comes into contact with a filter medium,
(c) said filter medium having affixed to its surface specialized cell-surface receptors intended to engage a quantity of a unique type of specific cell-surface receptor located on the surface of said specific target cells,
(d) said filter medium having affixed to its surface a quantity of FasL cell-surface receptors intended to engage Fas cell-surface receptors on said specific target cells,
(e) said blood exits said chamber at a different portal than where said blood entered said chamber,
(f) said filter medium is retained inside said chamber,
whereby said cell-surface receptors unique to said specific target cells and located on the surface of said specific target cells are intended to come in contact with said specialized cell-surface receptors found on the surface of said filter medium contained inside said chamber,
whereby said unique type of specific cell-surface receptors unique to said specific target cells and located on the surface of said specific target cells are intended to engage said specialized cell-surface receptors, followed by FasL cell-surface receptors found on the surface of said filter medium contained inside said chamber engaging Fas cell-surface receptors located on the specific target cells with the intention of triggering apoptosis inside said specific target cells for the purpose of terminating said specific target cells,
whereby the said specific target cells could be T-Helper cells infected with HIV genome circulating in the blood, or specific target cells could be any cell circulating in the blood acting as a host infected with a virus, or specific target cells could be any cell circulating in the blood acting as a host infected with a parasite, or specific target cells could be cancer cells circulating in the blood,
whereby said chamber may be used as a medical device that is inserted into a blood vessel in a body or may be used as a medical device to outside the body.

19. The medical device in claim 18 wherein said filter medium selected from the group consisting of a quantity of lipid bilayer sheets, a quantity of lipid bilayer strips, a quantity of lipid bilayer spheres, a quantity of virus-like structures, a quantity of hypoallergenic surfaces fashioned in the shape of a sheet capable of acting as a base on which can be affixed said cell-surface receptors to support the functional expression of a quantity of said cell-surface receptors, a quantity of hypoallergenic surfaces fashioned in the shape of a strip capable of acting as a base on which can be affixed said cell-surface receptors to support the functional expression of a quantity of said cell-surface receptors, and a quantity of hypoallergenic surfaces fashioned in the shape of a sphere capable of acting as a base on which can be affixed said cell-surface receptors to support the functional expression of a quantity of said cell-surface receptors.

20. The medical device in claim 18 wherein said quantity of specialized cell-surface receptors affixed to said filter medium, that engage a quantity of a unique type of specific cell-surface receptors located on the surface of said specific target cells, are physically constructed to be more prominent on the surface of said filter medium to facilitate that said specialized cell-surface receptors are engaged by said unique type of specific cell-surface receptors located on said specific target cells before a quantity of said FasL cell-surface receptors affixed to said filter medium are allowed the opportunity to engage a quantity of Fas cell-surface receptors located on said specific target cells,
whereby the action of said unique type of specific cell-surface receptors located on said specific target cells engaging said specialized cell-surface receptors affixed to the surface of said filter medium followed by a quantity of said FasL cell-surface receptors affixed to said filter medium engaging a quantity of Fas cell-surface receptors located on said specific target cells in the manner described will trigger apoptosis inside said specific target cells for the purpose of terminating said specific target cells,
whereby the intention that said specialized cell-surface receptor affixed to the filter medium must be engaged by a unique type of specific cell-surface receptor found on said specific target cells before said FasL cell-surface receptors affixed to said filter medium are able to engage said Fas cell-surface receptors located on said specific target cells, facilitates that cells that do not carry a quantity of the unique type of specific cell-surface receptors found on said specific target cells, will not be harmed by transiting through said medical device.

21. A medical device, to terminate specific target cells as they transit through lymph, comprised of a chamber:
(a) where lymph enters said chamber at one portal,
(b) said lymph comes into contact with a filter medium,
(c) said filter medium having affixed to its surface specialized cell-surface receptors intended to engage a quantity of unique type of specific of cell-surface receptors located on the surface of said specific target cells,
(d) said filter medium having affixed to its surface a quantity of FasL cell-surface receptors intended to engage Fas cell-surface receptors on said specific target cells,
(e) said lymph exits said chamber at a different portal than where said lymph entered said chamber,
(f) said filter medium is retained inside said chamber,
whereby said cell-surface receptors unique to said specific target cells and located on the surface of said specific target cells are intended to come in contact with said specialized cell-surface receptors found on the surface of said filter medium contained inside said chamber,
whereby said unique type of specific cell-surface receptors unique to said specific target cells and located on the surface of said specific target cells are intended to engage said specialized cell-surface receptors, followed by FasL cell-surface receptors found on the surface of said filter medium contained inside said chamber engaging Fas cell-surface receptors located on the specific target cells with the intention of triggering apoptosis inside said specific target cells for the purpose of terminating said specific target cells,
whereby the said specific target cells could be T-Helper cells infected with HIV genome circulating in the lymph, or specific target cells could be any cells circulating in the lymph acting as a host infected with a virus, or specific target cells could be any cells circulating in the lymph acting as a host infected with a parasite, or specific target cells could be a cancer cells circulating in the lymph,
whereby said chamber may be used as a medical device that is inserted into a lymphatic vessel in a body.

22. The medical device in claim 21 wherein said filter medium selected from the group consisting of a quantity of lipid bilayer sheets, a quantity of lipid bilayer strips, a quantity of lipid bilayer spheres, a quantity of virus-like structures, a quantity of hypoallergenic surfaces fashioned in the shape of a sheet capable of acting as a base on which can be affixed said cell-surface receptors to support the functional expression of a quantity of said cell-surface receptors, a quantity of hypoallergenic surfaces fashioned in the shape of a strip capable of acting as a base on which can be affixed said cell-surface receptors to support the functional expression of a quantity of said cell-surface receptors, and a quantity of hypoallergenic surfaces fashioned in the shape of a sphere capable of acting as a base on which can be affixed said cell-surface receptors to support the functional expression of a quantity of said cell-surface receptors.

23. The medical device in claim 21 wherein said quantity of specialized cell-surface receptors affixed to said filter medium, that engage a quantity of a unique type of specific cell-surface receptors located on the surface of said specific target cells, are physically constructed to be more prominent on the surface of said filter medium to facilitate that said specialized cell-surface receptors are engaged by said unique type of specific cell-surface receptors located on said specific target cells before a quantity of said FasL cell-surface receptors affixed to said filter medium are allowed the opportunity to engage a quantity of Fas cell-surface receptors located on said specific target cells,
whereby the action of said unique type of specific cell-surface receptors located on said specific target cells engaging said specialized cell-surface receptors affixed to the surface of said filter medium followed by a quantity of said FasL cell-surface receptors affixed to said filter medium engaging a quantity of Fas cell-surface receptors located on said specific target cells in the manner described will trigger apoptosis inside said specific target cells for the purpose of terminating said specific target cells,
whereby the intention that said specialized cell-surface receptor affixed to the filter medium must be engaged by a unique type of specific cell-surface receptor found on said specific target cells before said FasL cell-surface receptors affixed to said filter medium are able to engage said Fas cell-surface receptors located on said specific target cells, facilitates that cells that do not carry a quantity of the unique type of specific cell-surface receptors found on said specific target cells, will not be harmed by transiting through said medical device.
